(19) United States
(12) Patent Application Publication
(10) Pub. No.: US 2022/0323089 A1
(43) Pub. Date: Oct. 13, 2022

(54) VIBRATION TRANSMISSION MEMBER, ULTRASOUND TREATMENT TOOL, AND METHOD OF MANUFACTURING VIBRATION TRANSMISSION MEMBER

(71) Applicant: OLYMPUS CORPORATION, Tokyo (JP)

(72) Inventors: Tsunetaka AKAGANE, Hachioji-shi (JP); Michito MATSUOKA, Hachioji-shi (JP)

(73) Assignee: OLYMPUS CORPORATION, Tokyo (JP)

(21) Appl. No.: 17/849,098

(22) Filed: Jun. 24, 2022

Related U.S. Application Data
(63) Continuation of application No. PCT/JP2020/025634, filed on Jun. 30, 2020.

(51) Int. Cl. A61B 17/28

(52) U.S. Cl. CPC A61B 17/28; A61B 2017/00681

(57) ABSTRACT

A vibration transmission member includes: a main body portion configured to transmit ultrasound vibration; a treatment portion that is provided at a distal end of the main body portion, the treatment portion being configured to apply ultrasound vibration to a living tissue; a coating portion that is made of an electrically insulating material containing a first resin, the coating portion being configured to cover a part of a surface of the treatment portion; and a mold portion that is made of an electrically insulating material containing a second resin different from the first resin, and that is fused to at least a part of the coating portion.

### CROSS-REFERENCE TO RELATED APPLICATION

This application is a continuation of International Application No. PCT/JP2020/025634, filed on Jun. 30, 2020, the entire contents of which are incorporated herein by reference.

### BACKGROUND

### 1. Technical Field

The present disclosure relates to a vibration transmission member, an ultrasound treatment tool, and a method of manufacturing a vibration transmission member.

### 2. Related Art

In the related art, there has been known an ultrasound treatment tool that treats a treatment target site (hereinafter, described as a target site) in a living tissue by applying ultrasound vibration to the target site.

The ultrasound treatment tool includes a vibration transmission member that transmits ultrasound vibration from a proximal end toward a distal end and applies ultrasound vibration to a target site from a treatment surface provided at the distal end to treat the target site.

Meanwhile, in a case where a target site is treated by application of ultrasound vibration, the temperature of the outer surface of the vibration transmission member other than the treatment surface also increases. Then, in a case where the outer surface comes into contact with a site other than the target site in the living tissue in a state where the temperature of the outer surface is high, an unintended effect is exerted on the living tissue.

In the ultrasound treatment tool described above, a coating portion is formed on an outer surface of a vibration transmission member and a mold portion is integrally formed on the coating portion to improve heat insulation performance in order to avoid an unintended action on a living tissue.

### SUMMARY

In some embodiments, a vibration transmission member includes: a main body portion configured to transmit ultrasound vibration from a proximal end of the main body portion toward a distal end of the main body portion; a treatment portion that is provided at the distal end of the main body portion, the treatment portion being configured to apply ultrasound vibration to a living tissue; a coating portion that is made of an electrically insulating material containing a first resin, the coating portion being configured to cover a part of a surface of the treatment portion; and a mold portion that is made of an electrically insulating material containing a second resin different from the first resin, and that is fused to at least a part of the coating portion, the second resin including a base material in which an ether group and a ketone group are linked by a specific sequence, and a low Young's modulus material having a Young's modulus lower than a Young's modulus of the base material.

In some embodiments, provided is a method of manufacturing a vibration transmission member including a main body portion configured to apply ultrasound vibration from a treatment portion provided at a distal end of the main body portion to a living tissue. The method includes: forming a coating portion covering a part of a surface of the treatment portion by using an electrically insulating material containing a first resin; and fusing a mold portion to at least a part of the coating portion by bonding with the coating portion by heat treatment by using an electrically insulating material containing a second resin different from the first resin.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a view illustrating a treatment apparatus according to an exemplary embodiment;

FIG. 2 is a cross-sectional view illustrating a vibrator unit;

FIG. 3 is a perspective view illustrating a vibration transmission member;

FIG. 4 is a cross-sectional view illustrating a portion on a distal end side of the vibration transmission member;

FIG. 5 is a cross-sectional view illustrating a portion on the distal end side of the vibration transmission member;

FIG. 6 is a flowchart illustrating a method of manufacturing the vibration transmission member;

FIG. 7 is a view for explaining step S3;

FIG. 8 is a view for explaining step S3;

FIG. 9 is a view illustrating step S3;

FIG. 10 is a view for describing a modification of step S3 according to exemplary embodiments;

FIG. 11 is a view for describing a modification of step S3 according to exemplary embodiments; and

FIG. 12 is a view for describing a modification of step S3 according to exemplary embodiments.

### DETAILED DESCRIPTION

Hereinafter, modes for carrying out the disclosure (hereinafter, embodiments) will be described with reference to the drawings. Note that the disclosure is not limited by the embodiments described below. Furthermore, in the description of the drawings, the same components are denoted by the same reference signs.

Schematic Configuration of Treatment Apparatus

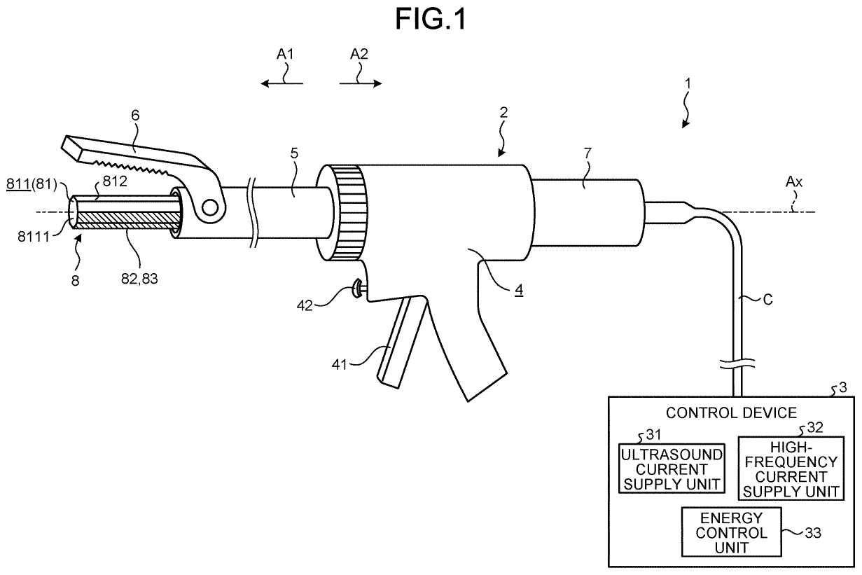

FIG. 1 is a view illustrating a treatment apparatus 1 according to an exemplary embodiment.

The treatment apparatus 1 applies ultrasound energy and high-frequency energy to a treatment target site (hereinafter, described as a target site) in a living tissue to treat the target site. Here, the treatment means, for example, coagulation and incision of a target site. As illustrated in FIG. 1, the treatment apparatus 1 includes an ultrasound treatment tool 2 and a control device 3.

The ultrasound treatment tool 2 is, for example, a medical treatment tool using a bolted Langevin type vibrator (BLT) for treating a target site in a state of passing through the abdominal wall. As illustrated in FIG. 1, the ultrasound treatment tool 2 includes a handle 4, a sheath 5, a jaw 6, a vibrator unit 7, and a vibration transmission member 8.

The handle 4 is a portion held by the operator's hand. Then, as illustrated in FIG. 1, the handle 4 is provided with an operation knob 41 and an operation button 42.

The sheath 5 has a cylindrical shape. Hereinafter, the central axis of the sheath 5 is referred to as a central axis Ax (FIG. 1). In addition, hereinafter, one side along the central axis Ax is referred to as a distal end side A1 (FIG. 1), and the other side is referred to as a proximal end side A2 (FIG. 1). Then, the sheath 5 is attached to the handle 4 by inserting a part of the proximal end side A2 into the handle 4 from the distal end side A1 of the handle 4.

The jaw 6 is rotatably attached to an end portion of the sheath 5 on the distal end side A1, and grips a target site between the jaw 6 and a portion of the vibration transmission member 8 on the distal end side A1. Note that an opening/closing mechanism (not illustrated) that opens and closes the jaw 6 with respect to the portion on the distal end side A1 of the vibration transmission member 8 according to the operation of the operation knob 41 by the operator is provided inside the handle 4 and the sheath 5 described above.

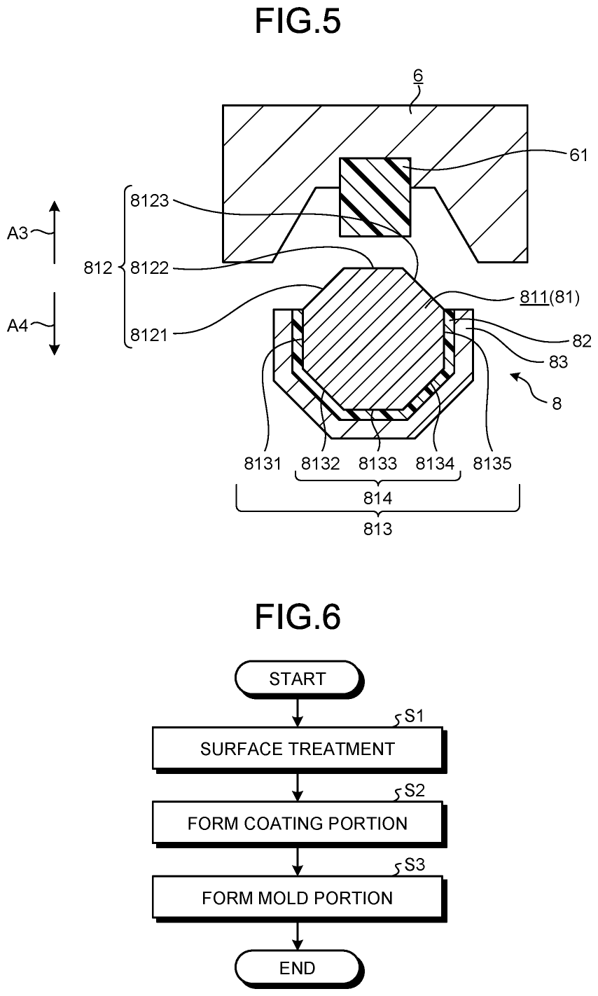

In the jaw 6, a pad 61 (see FIG. 5) made of resin is attached to a surface facing the vibration transmission member 8. Since the pad 61 has an electrically insulating property, it has a function of preventing a short circuit between the jaw 6 and the vibration transmission member 8. In addition, the pad 61 has a function of preventing the vibration transmission member 8 that is ultrasonically vibrating from being damaged by colliding with the jaw 6 when the incision of the target site by the ultrasound vibration is completed.

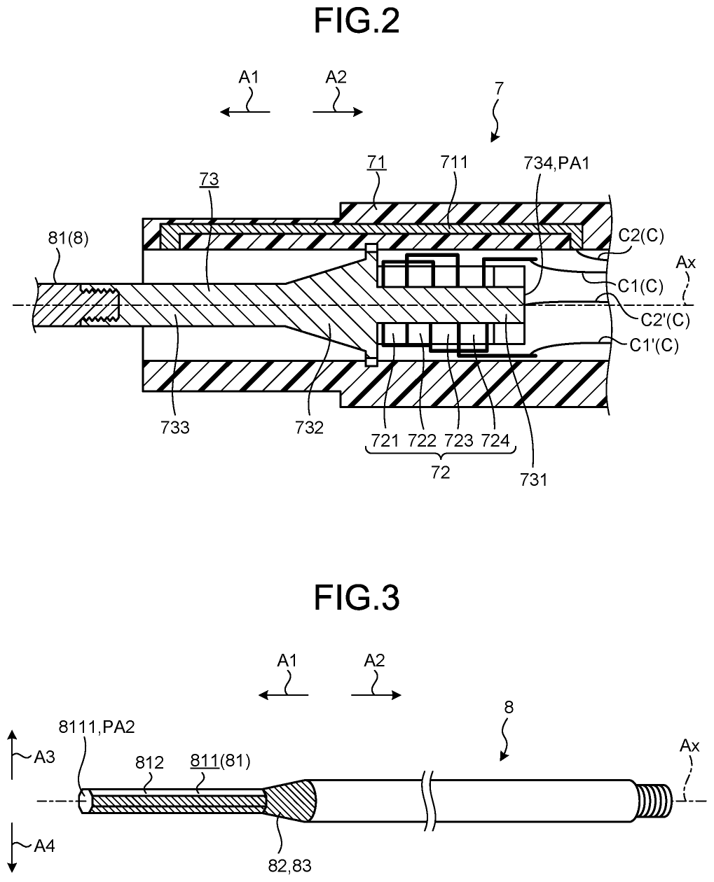

FIG. 2 is a cross-sectional view illustrating the vibrator unit 7. Specifically, FIG. 2 is a cross-sectional view of the vibrator unit 7 cut along a plane including the central axis Ax.

As illustrated in FIG. 2, the vibrator unit 7 includes a vibrator case 71, an ultrasound vibrator 72, and a horn 73.

The vibrator case 71 extends linearly along the central axis Ax, and is attached to the handle 4 by inserting a part of the distal end side A1 into the handle 4 from the proximal end side A2 of the handle 4. Then, when the vibrator case 71 is attached to the handle 4, the end portion on the distal end side A1 is connected to the end portion on the proximal end side A2 of the sheath 5.

The ultrasound vibrator 72 is housed inside the vibrator case 71 and generates ultrasound vibration under the control of the control device 3. In the present embodiment, the ultrasound vibration is longitudinal vibration that vibrates in a direction along the central axis Ax. As illustrated in FIG. 2, the ultrasound vibrator 72 is a BLT including a plurality of piezoelectric elements 721 to 724 stacked along the central axis Ax. In the present embodiment, the piezoelectric elements are composed of four piezoelectric elements 721 to 724, but the number of piezoelectric elements is not limited to four, and other numbers may be used.

The horn 73 is housed inside the vibrator case 71 and enlarges the amplitude of the ultrasound vibration generated by the ultrasound vibrator 72. The horn 73 has an elongated shape extending linearly along the central axis Ax. Then, as illustrated in FIG. 2, the horn 73 has a configuration in which a first attachment portion 731, a cross-sectional area change portion 732, and a second attachment portion 733 are arranged from the proximal end side A2 to the distal end side A1.

The first attachment portion 731 is a portion to which the ultrasound vibrator 72 is attached.

The cross-sectional area change portion 732 has a shape in which the cross-sectional area decreases toward the distal end side A1, and is a portion that enlarges the amplitude of the ultrasound vibration.

The second attachment portion 733 is a portion to which the vibration transmission member 8 is attached.

The vibration transmission member 8 has an elongated shape extending linearly along the central axis Ax, and is inserted into the sheath 5 in a state where a portion on the distal end side A1 protrudes to the outside as illustrated in FIG. 1. In addition, as illustrated in FIG. 2, an end portion of the vibration transmission member 8 on the proximal end side A2 is connected to the second attachment portion 733. Then, the vibration transmission member 8 transmits the ultrasound vibration generated by the ultrasound vibrator 72 and having passed through the horn 73 from the end portion on the proximal end side A2 to the end portion on the distal end side A1, and applies the ultrasound vibration to the target site gripped between the end portion on the distal end side A1 and the jaw 6 to treat the target site. That is, the target site is treated by application of ultrasound energy from the end portion on the distal end side A1. Note that the detailed configuration of the vibration transmission member 8 will be described later.

The control device 3 is electrically connected to the ultrasound treatment tool 2 by an electric cable C (FIG. 1), and comprehensively controls the operation of the ultrasound treatment tool 2. As illustrated in FIG. 1, the control device 3 includes an ultrasound current supply unit 31, a high-frequency current supply unit 32, and an energy control unit 33.

Here, as illustrated in FIG. 2, a pair of vibrator lead wires C1 and C1′ constituting the electric cable C is bonded to the ultrasound vibrator 72.

Then, the ultrasound current supply unit 31 supplies AC power to the ultrasound vibrator 72 via the pair of vibrator lead wires C1 and C1′ under the control of the energy control unit 33. As a result, the ultrasound vibrator 72 generates ultrasound vibration.

Here, as illustrated in FIG. 2, the vibrator case 71 is provided with a first conductive portion 711 extending from the end portion on the proximal end side A2 to the end portion on the distal end side A1. In addition, although not specifically illustrated, the sheath 5 is provided with a second conductive portion that extends from the end portion on the proximal end side A2 to the end portion on the distal end side A1 and electrically connects the first conductive portion 711 to the jaw 6. Furthermore, a high-frequency lead wire C2 constituting the electric cable C is bonded to the end portion of the first conductive portion 711 on the proximal end side A2. Furthermore, a high-frequency lead wire C2′ constituting the electric cable C is bonded to the first attachment portion 731.

Then, under the control of the energy control unit 33, the high-frequency current supply unit 32 supplies a high-frequency current between the jaw 6 and the vibration transmission member 8 via the pair of high-frequency lead wires C2 and C2′, the first conductive portion 711, the second conductive portion, and the horn 73. As a result, a high-frequency current flows through the target site gripped between the jaw 6 and the portion of the vibration transmission member 8 on the distal end side A1. That is, high-frequency energy is applied to the target site. Then, Joule heat is generated by the high-frequency current flowing through the target site, and the target portion is treated.

As described above, the jaw 6 and the vibration transmission member 8 also function as a high-frequency electrode. In other words, the ultrasound treatment tool 2 also functions as a bipolar treatment tool by the jaw 6 and the vibration transmission member 8 functioning as a pair of high-frequency electrodes.

The energy control unit 33 is, for example, a central processing unit (CPU), a field-programmable gate array (FPGA), or the like, and controls the operation of the ultrasound current supply unit 31 and the high-frequency current supply unit 32 in a case where the operation button 42 is pressed by the operator.

Configuration of Vibration Transmission Member

Next, a detailed configuration of the above-described vibration transmission member 8 will be described.

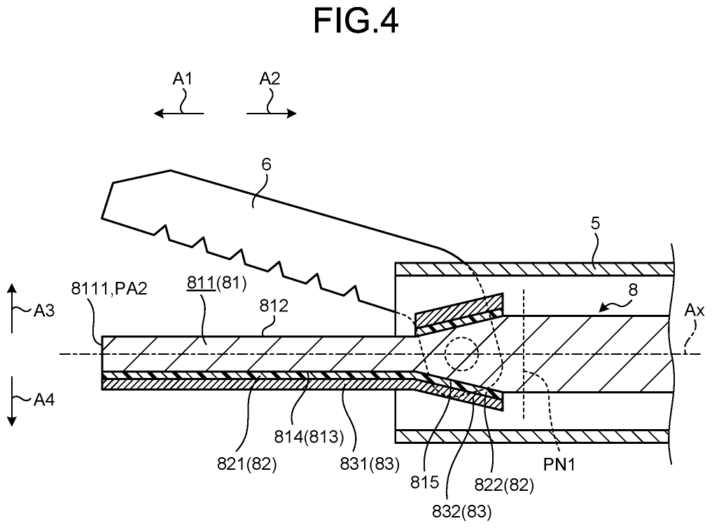

FIG. 3 is a perspective view illustrating the vibration transmission member 8. FIG. 4 and FIG. 5 are a cross-sectional view illustrating a portion on the distal end side A1 of the vibration transmission member 8. Specifically, FIG. 4 is a cross-sectional view of the vibration transmission member 8 taken along a plane including the central axis Ax and passing through a treatment surface 812. FIG. 5 is a cross-sectional view of the vibration transmission member 8 and the jaw 6 taken along a plane orthogonal to the central axis Ax and passing through a treatment portion 811.

In the following description of the configuration of the vibration transmission member 8, the side of the jaw 6 (upper side in FIGS. 3 to 5) is referred to as a jaw side A3 (FIGS. 3 to 5), and the side away from the jaw 6 (lower side in FIGS. 3 to 5) is referred to as a back surface side A4 (FIGS. 3 to 5).

As illustrated in FIGS. 3 to 5, the vibration transmission member 8 includes a main body portion 81, a coating portion 82, and a mold portion 83. Note that, in FIG. 3, hatching is applied to positions where the coating portion 82 and the mold portion 83 are provided for convenience of description.

The main body portion 81 is formed of, for example, a titanium alloy or the like, and has an elongated shape extending linearly along the central axis Ax as illustrated in FIG. 3. Then, the main body portion 81 transmits the ultrasound vibration generated by the ultrasound vibrator 72 and having passed through the horn 73 from the end portion on the proximal end side A2 to the end portion on the distal end side A1.

Here, the main body portion 81, the horn 73, and the ultrasound vibrator 72 are one vibrator that performs longitudinal vibration by ultrasound vibration at a predetermined resonance frequency generated by the ultrasound vibrator 72. Therefore, a proximal end surface 734 (FIG. 2) of the horn 73 is located at a most proximal antinode position PA1 (FIG. 2) located on the most proximal end side A2 among the positions of antinodes of the longitudinal vibration. In addition, a distal end surface 8111 (FIGS. 3 and 4) of the main body portion 81 is located at a most distal antinode position PA2 (FIGS. 3 and 4) located on the most distal end side A1 among the positions of antinodes of the longitudinal vibration. Note that the longitudinal vibration has a frequency of, for example, 47 kHz and an amplitude of, for example, 80 μm at the most distal antinode position PA2.

In the main body portion 81, the end portion on the distal end side A1 functions as the treatment portion 811 (FIGS. 1 and 3 to 5) that treats a target site in a state of gripping the target site with the jaw 6. The treatment portion 811 is a portion located on the distal end side A1 with respect to a most distal end node position PN1 (FIG. 4) located on the most distal end side A1 among the node positions of the longitudinal vibration. Although not specifically illustrated, a lining for supporting the main body portion 81 with respect to the sheath 5 is provided between the main body portion 81 and the sheath 5 at the most distal end node position PN1. Then, a part of the treatment portion 811 protrudes from the sheath 5 toward the distal end side A1.

In the present embodiment, as illustrated in FIG. 5, the treatment portion 811 has an octagonal cross section, and is positioned in a posture in which three sides 8121 to 8123 of the octagonal cross section face the jaw side A3. Then, the surfaces corresponding to the three sides 8121 to 8123 come into contact with the target site in a state where the target site is gripped between the treatment portion 811 and the jaw 6 and function as the treatment surface 812 for treating the target site. In addition, of surfaces 813 corresponding to the five sides 8131 to 8135 excluding the treatment surface 812 in the octagonal cross-sectional shape, the surfaces corresponding to the three sides 8132 to 8134 are on an opposite side of the treatment surface 812. That is, the surface corresponding to the three sides 8132 to 8134 is positioned in a posture facing the back surface side A4 and corresponds to a back surface 814.

The coating portion 82 is a portion covering a part of the surface of the treatment portion 811, and is formed of a material having electrical insulation and thermal conductivity lower than that of the main body portion 81. In the present embodiment, the coating portion 82 is formed of a material containing a first resin as a main component and a linear expansion adjustment filler having a linear expansion coefficient smaller than that of the main component. Note that the first resin will be described later. In addition, examples of the linear expansion adjustment filler include mica. Then, the linear expansion adjustment filler is preferably contained in an amount of 0.1 mass % or more and 50 mass % or less.

Here, the region where the coating portion 82 is provided is as follows.

The coating portion 82 is provided in a region excluding the distal end surface 8111 and the treatment surface 812 in the treatment portion 811. That is, the coating portion 82 is provided on the surface 813 including the back surface 814. Hereinafter, in the coating portion 82, a portion provided on the surface 813 is referred to as a first coating portion 821 (FIG. 4). In addition, the coating portion 82 includes a second coating portion 822 (FIG. 4) connected to the proximal end side A2 with respect to the first coating portion 821. The second coating portion 822 is located on the proximal end side A2 in the treatment portion 811 and is provided on a surface 815 (FIG. 4) positioned inside the sheath 5. Then, the second coating portion 822 extends over the entire circumference in the circumferential direction centered on the central axis Ax on the surface 815.

The mold portion 83 is integrally formed on the coating portion 82 by, for example, insert molding or outsert molding using a material having electrical insulation and thermal conductivity lower than that of the main body portion 81. In the present embodiment, the mold portion 83 is formed of a material containing a second resin different from the first resin constituting the coating portion 82 as a main component and a glass filler contained in the main component. Note that the second resin will be described later. In addition, the glass filler is preferably contained in an amount of 10 mass % or more and 40 mass % or less.

Here, the region where the mold portion 83 is provided is the same as the region where the coating portion 82 is provided. That is, as illustrated in FIG. 4, the mold portion 83 includes a first mold portion 831 provided on the entire first coating portion 821 and a second mold portion 832 provided on the entire second coating portion 822. Note that the mold portion 83 may be provided only on a part of the coating portion 82 without being provided on the entire coating portion 82.

First and Second Resins

Next, the above first and second resins will be described.

The first resin is a polymer of any of three kinds of polymers of polyether ether ketone (PEEK)

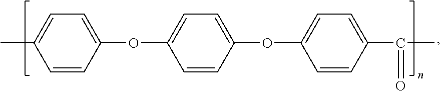

polyether ketone (PEK)

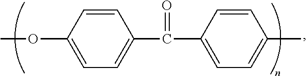

and
polyether ketone ether ketone ketone (PEKEKK)

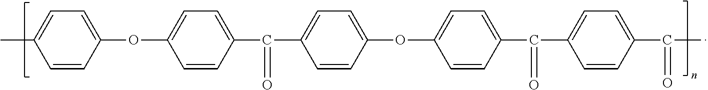

In the first resin, ether groups and ketone groups are linked by a first sequence (the respective monomer units forming ether groups or ketone groups are present in the polymer backbone in a first order).

On the other hand, the second resin is a polymer different from the first resin among the three kinds of polymers. These three kinds of polymers are polymers in which ether groups and ketone groups are linked by a specific sequence. Then, in the three kinds of polymers, the specific sequences are different from each other. That is, the ether groups and ketone groups are linked by a second sequence different from the first sequence (the respective monomer units forming ether groups or ketone groups are present in the polymer backbone in a second order.

Table 1 below shows the Young's modulus, the melting point, and the compressive strength of the three polymers.

TABLE 1

| | PEEK | PEK | PEKEKK |
|---|---|---|---|
| Young's modulus (GPa) | 3.7 | 3.7 | 4.3 |
| Melting point (° C.) | 343 | 373 | 387 |
| Compressive strength (MPa) | 120 | 140 | 145 |
| (23° C.) | | | |

In the present embodiment, PEEK in which ether groups and ketone groups are linked by a first sequence is adopted as the first resin. On the other hand, PEK or PEKEKK in which ether groups and ketone groups are linked by a second sequence different from the first sequence is adopted as the second resin. That is, in a case where PEK is adopted as the second resin, the Young's modulus of the second resin is equal to or less than the Young's modulus of the first resin (PEEK). In addition, the melting point of the second resin (PEK or PEKEKK) is higher than the melting point of the first resin (PEEK). Furthermore, the compressive strength of the second resin (PEK or PEKEKK) is higher than the compressive strength of the first resin (PEEK).

Method of Manufacturing Vibration Transmission Member

Next, a method of manufacturing the above-described vibration transmission member 8 will be described.

FIG. 6 is a flowchart illustrating a method of manufacturing the vibration transmission member 8.

First, the worker performs a surface treatment on the surfaces 813 and 815 in order to enhance the adhesion strength of the coating portion 82 to the surfaces 813 and 815 (step S1).

Specifically, as the surface treatment, a surface treatment for increasing the surface roughness by sandblasting can be exemplified.

After step S1, the worker forms the coating portion 82 on the surfaces 813 and 815 where surface treatment has been applied (step S2).

Specifically, in step S2, the worker sprays a liquid contained in the linear expansion adjustment filler (mica) in a scaly shape onto the surfaces 813 and 815 with respect to the granular first resin (PEEK). Then, the worker heats the liquid. As a result, the coating portion 82 is formed. The thickness of the coating portion 82 is about 15 to 80 μm.

In the surface treatment in step S1, the oxide film is removed from the surfaces 813 and 815, and an anchor effect and a stress effect from the uneven surface are applied between the surfaces 813 and 815 and the oxide film. As a result, the adhesion strength of the coating portion 82 to the surfaces 813 and 815 is enhanced.

After step S2, the worker forms the mold portion 83 on the coating portion 82 (step S3).

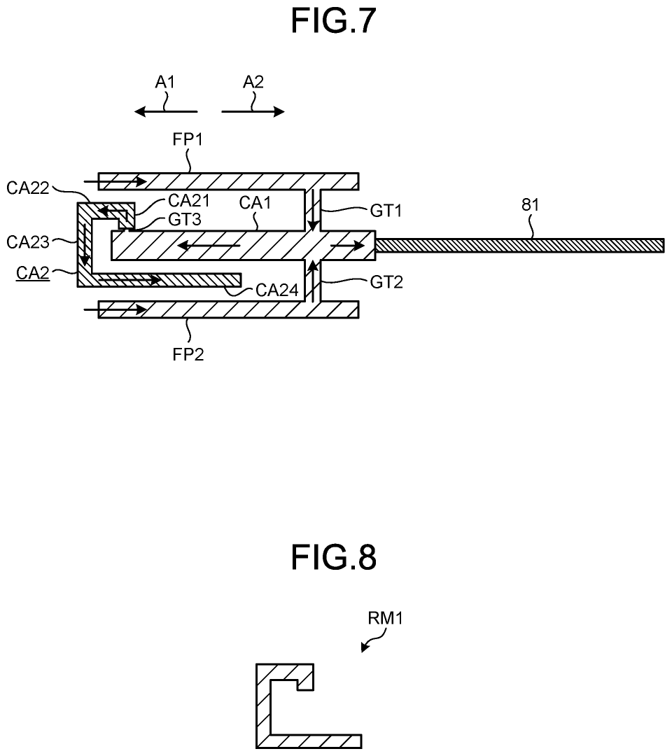

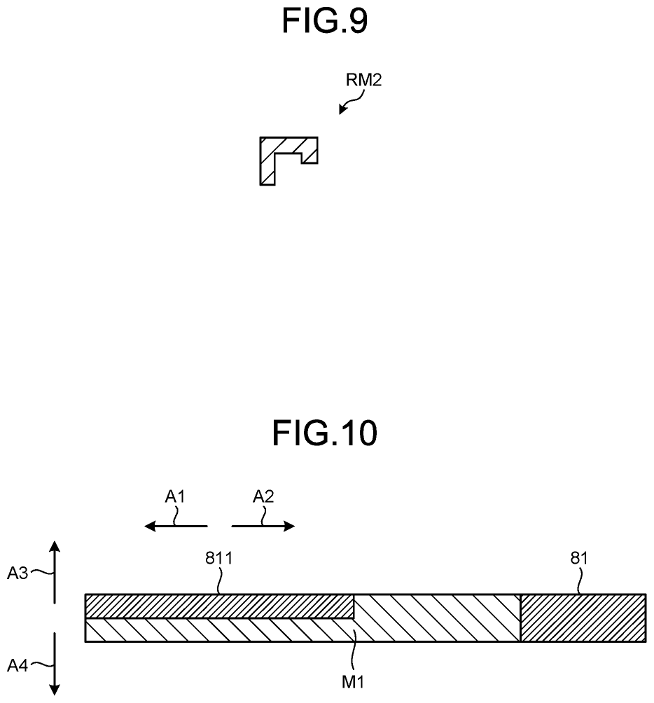

FIGS. 7 to 9 are views illustrating step S3. Specifically, FIG. 7 is a view illustrating the main body portion 81, a first cavity CA1, flow paths FP1 and FP2, gates GT1 to GT3, and a second cavity CA2 provided in the mold (not illustrated) from the back surface side A4. In FIG. 7, the flow of the material constituting the molten mold portion 83 is represented by arrows. FIGS. 8 and 9 are views illustrating an example (reference molded bodies RM1 and RM2) of the molded body formed in the second cavity CA2.

Here, the first cavity CA1 is a cavity for forming the mold portion 83 on the coating portion 82, and has shapes corresponding to the outer shapes of the main body portion 81, the coating portion 82, and the mold portion 83. In addition, the flow paths FP1 and FP2 are flow paths of a sprue, a runner, or the like extending substantially parallel to the first cavity CA1 at a position separated from the first cavity CA1. Furthermore, the gates GT1 and GT2 are gates that communicate the first cavity CA1 with the flow paths FP1 and FP2 at the same position in the direction along the central axis Ax. Then, the flow paths FP1 and FP2 and the gates GT1 and GT2 guide the material constituting the molten mold portion 83 toward the first cavity CA1. Furthermore, the second cavity CA2 communicates with the first cavity CA1 through the gate GT3 on the distal end side A1 of the first cavity CA1, and is used to evaluate the formed mold portion 83. More specifically, in the second cavity CA2, linear cavities CA21 to CA24 are connected in series while being substantially orthogonal to each other.

Specifically, in step S3, the worker installs the main body portion 81 on which the coating portion 82 is formed in the first cavity CA1. Next, the temperature of the mold (not illustrated) is set to a predetermined temperature. Here, the predetermined temperature can be, for example, a temperature equal to or higher than the glass transition point and equal to or lower than the melting point of the material constituting the mold portion 83. Next, the material constituting the molten mold portion 83 is injected into the first cavity CA1 after following the path of the flow paths FP1 and FP2 to the gates GT1 and GT2. As a result, the material constituting the molten mold portion 83 flows toward the distal end side A1 and the proximal end side A2 in the first cavity CA1. Then, the mold portion 83 is molded. The thickness of the mold portion 83 is about 300 μm.

In the present embodiment, the gates GT1 and GT2 are provided at the same position in the direction along the central axis Ax, in a manner that the molten material is prevented from joining in the direction along the central axis Ax in the first cavity CA1. That is, since the merged position is a weldline of which the strength decreases, a structure in which the weldline is not formed is adopted.

When the material constituting the molten mold portion 83 is injected into a mold (the first cavity CA1, the flow paths FP1 and FP2, the gates GT1 to GT3, and the second cavity CA2), it is necessary to avoid a decrease in temperature of the molten material due to the mold. Therefore, it is preferable to set the injection pressure and the injection rate at the time of injecting the molten material into the mold to be higher than the general injection pressure and injection rate.

Then, in a case where an injection pressure or an injection rate at the time of injecting the molten material into the mold is appropriate, the molten material flows into the second cavity CA2 through the gate GT3 as described below.

That is, the molten material flows into the cavity CA24 after following the path from the cavity CA21 to the cavity CA22 to the cavity CA23 in the second cavity CA2. As a result, the reference molded body RM1 illustrated in FIG. 8 is formed. That is, by forming the reference molded body RM1, it can be determined that the quality of the formed mold portion 83 is good.

On the other hand, in a case where the injection pressure and the injection rate are not appropriate, the molten material flows into the second cavity CA2 through the gate GT3 as follows.

That is, the molten material flows only to the cavity CA23 after following the path from the cavity CA21 to the cavity CA22 in the second cavity CA2. As a result, the reference molded body RM2 illustrated in FIG. 9 is formed. That is, by forming the reference molded body RM2, it can be determined that the quality of the formed mold portion 83 is not good.

As described above, the quality of the formed mold portion 83 can be evaluated by the length of the molded body formed in the second cavity CA2.

According to the embodiment described above, the following effects are obtained.

In the vibration transmission member 8 according to the present embodiment, the coating portion 82 is made of a material containing the first resin (PEEK). On the other hand, the mold portion 83 is made of a material containing the second resin (PEK or PEKEKK) different from the first resin.

Therefore, the adhesion strength of the mold portion 83 to the coating portion 82 can be enhanced by the intermolecular force exerted by the ether group and the ketone group. In addition, since the melting point and the compressive strength of the second resin are higher than those of the first resin (see Table 1), the mold portion 83 can have high heat resistance and high strength.

Therefore, even when the mold portion 83 is formed thick, peeling of the mold portion 83 from the coating portion 82 and destruction of the mold portion 83 can be suppressed, and durability of the mold portion 83 can be improved.

Next, another exemplary embodiment will be described. In the following description, the same reference signs are given to the same configurations as those of the embodiment described above, and a detailed description will be omitted or simplified.

In the present embodiment, only the second resin described in the above embodiment is different.

Specifically, the second resin according to the present embodiment includes a base material made of PEK or PEKEKK and a low Young's modulus material having a Young's modulus lower than that of the base material.

Examples of the low Young's modulus material include polyacetal (POM), polyphenylene sulfide (PPS), polyether sulfone (PES), polysulfone (PSU), polyether imide (PEI), polytetrafluoroethylene (PTFE), tetrafluoroethylene-perfluoroalkyl vinyl ether copolymer (PFA), polychlorotrifluoroethylene (PCTFE), and polyvinylidene fluoride (PVDF) as shown in Table 2 below.

In the present embodiment, any fluororesin of PTFE, PFA, PCTFE, and PVDF is adopted as the low Young's modulus material.

TABLE 2

| | Young's modulus (GPa) |
|---|---|
| POM | 2.59 |
| PPS | 3.3 |
| PES | 2.6 |
| PSU | 2.5 |
| PEI | 3 |
| PTFE | 0.5 |
| PFA | 0.33 |
| PCTFE | 1.5 |
| PVDF | 1.9 |

According to the present embodiment described above, the following effects are obtained in addition to the same effects as those of the embodiment described above.

The second resin constituting the mold portion 83 according to present embodiment is made of a base material made of PEK or PEKEKK and a low Young's modulus material having a Young's modulus lower than that of the base material. That is, the Young's modulus of the entire mold portion 83 can be reduced as compared with a case where the mold portion 83 is formed only of the base material. Therefore, the stress applied to the mold portion 83 by the ultrasound vibration can be reduced. As a result, peeling of the mold portion 83 from the coating portion 82 and destruction of the mold portion 83 can be suppressed, and durability of the mold portion 83 can be improved.

### Other Exemplary Embodiments

Although the embodiments for carrying out the disclosure have been described so far, the disclosure should not be limited only to the embodiments described above.

In the embodiments described above, the shape of the treatment portion 811 is not limited to the octagonal shape in cross section, and other shapes such as a circular shape in cross section may be adopted.

In the embodiments described above, both the ultrasound energy and the high-frequency energy are applied to the target site, but the disclosure is not limited to this, and only the ultrasound energy may be applied to the target site. In addition, at least one of high-frequency energy and thermal energy by a heater or the like and ultrasound energy may be applied to the target site.

In the embodiments described above, the first resin is not limited to any of the three polymers of PEEK, PEK, and PEKEKK, and other polymers having an ether group and a ketone group may be adopted. Note that the same applies to the second resin.

In the embodiments described above, the positions where the coating portion 82 and the mold portion 83 are provided are not limited to the positions described in the embodiments described above, and may be any position as long as the position includes the back surface 814.

FIGS. 10 to 12 are views for describing a modification of step S3 according to the embodiments described above. Specifically, FIGS. 10 to 12 are views of the main body portion 81 as viewed from the side.

In step S3 according to the present modification, when forming the mold portion 83, the worker sequentially molds first to third molded bodies M1 to M3 as illustrated in FIGS. 10 to 12.

As illustrated in FIG. 10, the first molded body M1 is a molded body to be molded first, and is a molded body molded over the entire coating portion 82.

As illustrated in FIG. 11, the second molded body M2 is a molded body molded into the entire portion corresponding to the first coating portion 821 and a part of the portion corresponding to the second coating portion 822 on the first molded body M1. Then, in the state of FIG. 11, a step ST1 is provided between the first molded body M1 and the second molded body M2. In addition, in the second molded body M2, a step ST2 is also provided between a portion corresponding to the first coating portion 821 and a portion corresponding to the second coating portion 822.

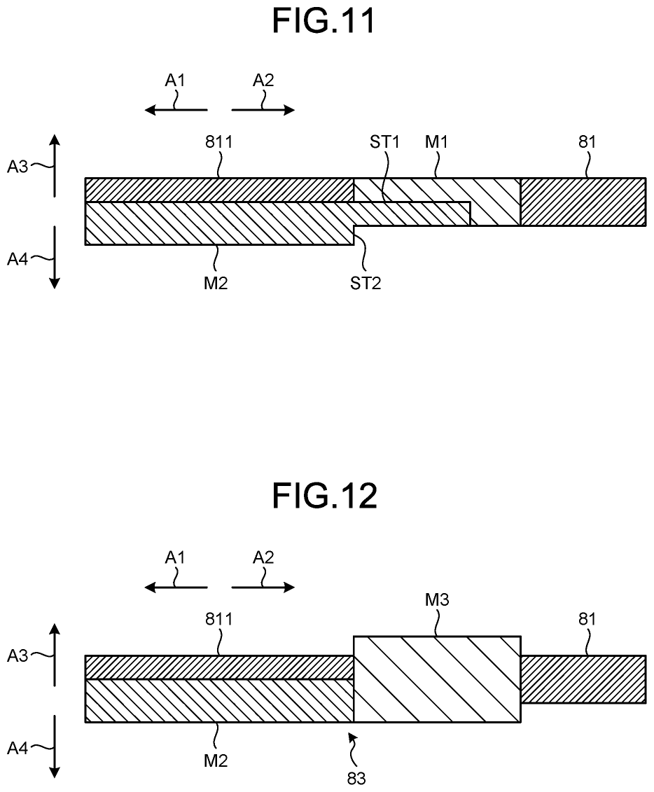

As illustrated in FIG. 12, the third molded body M3 is a molded body molded into the entire portion corresponding to the second coating portion 822 on the first and second molded bodies M1 and M2. As a result, the steps ST1 and ST2 are eliminated.

Then, the first to third molded bodies M1 to M3 are combined to form the mold portion 83.

According to the present modification described above, the following effects are obtained in addition to the effects similar to those of the embodiments described above.

In the present modification, in forming the mold portion 83, the first to third molded bodies M1 to M3 are sequentially formed. That is, since the first to third molded bodies M1 to M3 easy in temperature management are sequentially formed as compared with the configuration in which the mold portion 83 is collectively formed, it is possible to form the high-quality mold portion 83.

In addition, the third molded body M3 is formed on the first and second molded bodies M1 and M2 in a form eliminating the steps ST1 and ST2. Therefore, the adhesion strength of the third molded body M3 to the first and second molded bodies M1 and M2 can be enhanced.

According to the vibration transmission member, the ultrasound treatment tool, and the method of manufacturing a vibration transmission member according to the disclosure, the adhesion strength of the mold portion to the coating portion can be enhanced.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A vibration transmission member comprising:
a main body configured to transmit ultrasound vibration from a proximal end of the main body toward a distal end of the main body;
a treatment portion provided at the distal end of the main body, the treatment portion being configured to apply ultrasound vibration to a living tissue;
a coating covering a part of a surface of the treatment portion, the coating comprising a first electrically insulating material containing a first resin; and
a mold portion fused to at least a part of the coating, the mold portion comprising a second electrically insulating material containing a second resin different from the first resin.

2. The vibration transmission member according to claim 1, wherein
the treatment portion includes:
a treatment surface on one side of the treatment portion, the treatment surface being configured to apply ultrasound vibration to the living tissue, and
a back surface on an opposite side of the treatment portion, and
the coating and the mold portion are configured to cover a surface of the treatment portion that includes at least the back surface and excludes the treatment surface.

3. The vibration transmission member according to claim 1, wherein
the mold portion is fused to at least a part of the coating by insert molding.

4. The vibration transmission member according to claim 1, wherein
a Young's modulus of the second resin is equal to or less than a Young's modulus of the first resin.

5. The vibration transmission member according to claim 1, wherein
a melting point of the second resin is higher than a melting point of the first resin.

6. The vibration transmission member according to claim 1, wherein
a compressive strength of the second resin is higher than a compressive strength of the first resin.

7. The vibration transmission member according to claim 1, wherein
the first resin is a first polymer in which an ether group and a ketone group are linked by a first sequence, and
the second resin is a second polymer in which an ether group and a ketone group are linked by a second sequence different from the first sequence.

8. The vibration transmission member according to claim 7, wherein
the first polymer and the second polymer each are a polymer selected from the group consisting of polyether ether ketone, polyether ketone, and polyether ketone ether ketone ketone.

9. The vibration transmission member according to claim 1, wherein:
the coating has a thickness of 15 μm to 80 μm, and
the mold portion has a thickness of about 300 μm.

10. The vibration transmission member according to claim 1, wherein the second resin comprises:
a first material in which an ether group and a ketone group are linked, and
a second material having a Young's modulus lower than a Young's modulus of the first material.

11. The vibration transmission member according to claim 10, wherein
the second material is fluororesin.

12. An ultrasound treatment tool comprising:
a vibration transmission member configured to transmit ultrasound vibration; and
a jaw configured to grip a living tissue between the vibration transmission member and the jaw,
wherein the vibration transmission member comprises:
a main body configured to transmit ultrasound vibration from a proximal end of the main body portion toward a distal end of the main body portion,
a treatment portion provided at the distal end of the main body, the treatment portion being configured to apply ultrasound vibration to the living tissue,
a coating covering a part of a surface of the treatment portion, the coating comprising a first electrically insulating material containing a first resin, and
a mold portion fused to at least a part of the coating, the mold portion comprising a second electrically insulating material containing a second resin different from the first resin.

13. A method of manufacturing a vibration transmission member including a main body configured to apply ultrasound vibration from a treatment portion provided at a distal end of the main body to a living tissue, the method comprising:
forming a coating covering a part of a surface of the treatment portion by using a first electrically insulating material containing a first resin; and
fusing a mold portion to at least a part of the coating by bonding with the coating by heat treatment using a second electrically insulating material containing a second resin different from the first resin.
